(19) United States
(12) Patent Application Publication
(10) Pub. No.: US 2010/0028455 A1
(43) Pub. Date: Feb. 4, 2010

(54) NUTRITIONAL SUPPLEMENT

(76) Inventor: Neville Trevor Wittenbaker, Bayswater (AU)

Correspondence Address: BROOKS KUSHMAN P.C., 1000 TOWN CENTER, TWENTY-SECOND FLOOR, SOUTHFIELD, MI, 48075, US

(21) Appl. No.: 12/516,639

(22) PCT Filed: Nov. 28, 2007
(86) PCT No.: PCT/AU07/01825

§ 371 Date: May 28, 2009

(30) Foreign Application Priority Data
Nov. 29, 2006 (AU) 2006906674
Apr. 5, 2007 (AU) 2007201520

(51) Int. Cl. A61K 35/60; A61P 19/02; A61P 13/12; A61P 1/16

(52) U.S. Cl. 424/572

(57) ABSTRACT

A nutritional supplement derived from skeletal tissue found in bony and cartilaginous fish has been found to be useful in the treatment of mineral deficiencies, in particular calcium deficiency, and additionally, in the treatment of arthritis, kidney stones, and gout in animals, including humans. A nutritional supplement composition and a pharmaceutical composition containing the nutritional supplement are also provided. The nutritional supplement contains 300,000 to 400,000 mg/kg calcium, 100 to 150 mg/kg phosphorous, 10 to 20 mg/kg magnesium, and 1,500 to 2,000 mg/kg sodium.

### FIELD OF THE INVENTION

The present invention relates to a nutritional supplement, in particular to a nutritional supplement useful in the treatment of patients with a calcium deficiency, arthritis, kidney stones, and/or gout.

### SUMMARY OF THE INVENTION

The inventor has discovered that skeletal tissue found in fish is useful for the treatment of patients with a calcium deficiency. Further, the inventor has discovered that skeletal tissue found in fish is useful for the treatment of patients with arthritis, kidney stones, and/or gout.

Accordingly, in a first aspect of the invention there is provided a nutritional supplement derived from skeletal tissue found in fish.

In a second aspect of the invention there is provided a nutritional supplement composition comprising skeletal tissue found in fish and a carrier and/or excipient.

In a third aspect of the invention there is provided a pharmaceutical composition comprising skeletal tissue found in fish and a pharmaceutically acceptable carrier and/or excipient.

In a further aspect of the invention there is provided a method of treating a patient with a calcium deficiency comprising administering an effective amount of the nutritional supplement, nutritional supplement composition, or pharmaceutical composition as defined above.

In a yet further aspect of the invention there is provided a method of treating a patient with arthritis, kidney stones, and/or gout comprising administering an effective amount of the nutritional supplement, nutritional supplement composition, or pharmaceutical composition as defined above.

In one embodiment of the invention administering an effective amount of the nutritional supplement, nutritional supplement composition, or pharmaceutical composition comprises orally or parenterally administering an effective amount of the nutritional supplement, nutritional supplement composition, or pharmaceutical composition to the patient.

In another aspect of the invention there is provided a use of skeletal tissue found in fish in the manufacture of a medicament for the treatment of a patient with a calcium deficiency.

In still another aspect of the invention there is provided a use of skeletal tissue found in fish in the manufacture of a medicament for the treatment of a patient with arthritis, kidney stones, and/or gout.

### DETAILED DESCRIPTION OF THE PREFERRED EMBODIMENTS OF THE INVENTION

This invention relates to nutritional supplements, in particular to nutritional supplements derived from skeletal tissue found in fish, to nutritional supplement compositions containing said skeletal tissue, to pharmaceutical compositions containing said skeletal tissue and to the use of such nutritional supplements and compositions for the treatment of patients suffering from mineral deficiencies, arthritis, kidney stones, and/or gout.

Typically, the skeletal tissue is found in the skull of a fish, preferably configured as a pair of small deposits residing in the top of the skull. The skeletal tissue may be bony or cartilaginous. An illustrative example of the skeletal tissue includes, but is not limited to, otoliths which are found in the otic capsule, a cavity in the skull located below the brain. Otoliths are formed from polycrystalline calcium carbonate such as aragonite. It will be appreciated that in other embodiments of the invention, the skeletal tissue may also be formed from polycrystalline calcium carbonate.

In one embodiment of the invention the skeletal tissue may be found in bony fish. Preferably, the skeletal tissue is found in the skull of bony fish from the Gadidae family, more commonly known as the cod family. Illustrative examples of alternative families of cod or cod-like fish in which the skeletal tissue can be found include, but are not limited to, Melanonidae, Euclichthyidae, Merlucciidae, Stindachneriidae, and Macrouridae.

In alternative embodiments of the invention, the skeletal tissue is found in cartilaginous fish, such as sharks, rays, and skate.

Illustrative examples of families of bony or cartilaginous fish in which the skeletal tissue is known to be found include, but are not limited to:

Rhiniodontidae, Stegostimatidae, Ginglymostomatidae, Odontaspididae, Alopiidae, Heterodontidae, Lamnidae, Hexanchidae, Hemiscyllidae, Scyliorhinidae, Orectolobidae, Carcharhinidae, Spyrnidae, Pristidae, Rhynchobatidae, Rhinobatidae, Squatinidae, Urolophidae, Torpedinidae, Rajidae, Dasyatididae, Mobulidae, Gymnuridae, Myliobatidae, Chimaeridae, Elopidae, Albulidae, Chanidae, Meglopidae, Chirocentridae, clupeidae, Engraulidae, Muraenidae, Congridaw, Ophichthidae, Ariidae, Plotosidae, Synodotidae, Harpodontidae, Batrachoididae, Gobiescosidae, Antennariidae, Ophidiidae, Carapidae, Bythitidae, Exocoetidae, Hemirhamphidae, Belonidae, Atherinidae, Holocentridae, Monocentridae, Zeidae, Caproidae, Veliferidae, Fistularidae, Aulostomidae, Hoplichthidae, Dactylopteridae, Pegasidae, Centriscidae, Solenostomidae, Syngnathidae, Syngnathidae, Scorpaenidae, Aploactinidae, Triglidae, Platycephalidae, Serrandae, Grammistidae, Acanthoclinidae, Plesiopidae, Pseudochromidae, Centropomidae, Ambassidae, Glaucosomidae, Teraponidae, Sillaginidae, Priacanthidae, Apogonidae, Carangidae, Coryphaenidae, Malacanthidae, Pomatomidae, Rachycentridae, Echeneidae, Menidae, Leiognathidae, Lobotidae, Lutjanidae, Haemulidae, Caesionidae, Banjosidae, Lethrinidae, Sparidae, Menipteridae, Gerreidae, Mullidae, Scianidae, Monodactylidae, Leptobramidae, Pempheridae, Kyphosidae, Scorpidadae, Toxotidae, Scatophagidae, Ephippididae, Rhinoprenidae, Chaetodontidae, Pomacanthidae, Pomacentridae, Pentacerotidae, Cirrhitidae, Creediidae, Cepolidae, Congrodadidae, Notograptidae, Mugilidae, Polynemidae, Sphyraenidae, Labridae, Scaridae, Mugiloididae, Opistognathidae, Uranoscopidae, Blenniidae, Tripterygiidae, Callionumidae, Gobiidae, Eleotridae, Gobiodae, Zanclidae, Siganidae, Acanthuridae, Istiophoridae, Xiphiidae, Scombridae, Psetodidae, Bothidae, Pleuronectidae, Soleidae, Cynoglossidae, Percophididae, Centrolophidae, Nomeidae, Ariommatidae, Triodontidae, Triacanthodidae, Triacanthidae, Balistidae, Monacanthidae, Ostraciidae, Tetraodontidae, Diodontidae.

The nutritional supplement of the present invention is prepared by removing the skeletal tissue from the skull of the fish, and cleaning and grinding it to a fine powder. Typically, the skeletal tissue has a translucent milky white appearance, having a hardness of about 5 units on the Mohr scale. The average weight of the skeletal tissue is about 230 mg.

The resulting nutritional supplement contains 300,000 to 400,000 mg/kg calcium, 100 to 150 mg/kg phosphorous, 10 to 20 mg/kg magnesium, and 1,500 to 2,000 mg/kg sodium, and is suitable for consumption by mammals, including humans.

In particular, the above concentrations of calcium, phosphorous, and magnesium make the nutritional supplement of the present invention suitable for use in the treatment of mineral deficiencies, in particular calcium deficiency, in mammals, including humans, companion animals such as dogs and cats, domestic animals such as horses, cattle, and sheep, or zoo animals such as felids, canids, bovids, and ungulates.

The nutritional supplement may be ingested directly in a dry powder form or in admixture with solid or liquid food or beverages.

Additionally, the nutritional supplement may be combined with a carrier and/or excipient to produce a nutritional supplement composition. A wide number of acceptable carriers are known in the nutritional supplement arts, and the carrier can be any suitable carrier. The carrier need only be suitable for administration to animals, including humans, and be able to act as a carrier without substantially affecting the desired activity of the nutritional supplement. Also, the carrier(s) may be selected based upon the desired administration route and dosage form of the composition. For example, the nutritional supplement compositions according to the present invention are suitable for use in a variety of dosage forms, such as liquid form and solid form.

Further, a pharmaceutical composition may be prepared by combining the skeletal tissue found in fish with a pharmaceutically acceptable carrier and/or excipient.

The term “pharmaceutically acceptable” as used herein refers to pharmaceutically active agents or inert ingredients which are suitable for ingestion by animals, including humans, without undue toxicity, incompatibility, instability, irritation, allergic response, and the like, commensurate with a reasonable benefit/risk ratio.

The composition of the invention can be present in association with one or more pharmaceutically acceptable carriers and/or excipients and/or diluents and/or adjuvants, and if desired other active ingredients.

Compositions intended for oral use can be prepared according to any method known to the art for the manufacture of pharmaceutical compositions and such compositions can contain one or more sweetening agents, flavoring agents, coloring agents, preservative agents, stabilizers, buffers, dispersants, thickeners, solubilizing agents, and the like, as well as vitamins, minerals, coenzymes, organic or inorganic antioxidants or precursors thereof, additional nutritional supplements and/or herbal extracts, and other active ingredients.

Illustrative examples of sweetening agents which may be used in the compositions of the present invention include, but are not limited to, fructose, sucrose, sugar, dextrose, lactose, maltose, maltodextrins, corn syrup solids, honey solids, mannitol, sorbitol, xylitol, saccharin, aspartame, cyclamates, acesulfame K, neohesperidin dihydrochalcone, other super sweeteners, and mixtures thereof, which may be added to the carrier in amounts sufficiently low so as not to chemically interact with the nutritional supplement.

Illustrative examples of flavoring agents which may be used in the compositions of the present invention include, but are not limited to, peppermint, peppermint-menthol, eucalyptol, wintergreen, licorice, clove, cinnamon, spearmint, cherry, lemon, orange, lime, menthol and various combinations thereof.

The compositions of the present invention can be in a formulated in a suitable manner for oral use, for example, as tablets, troches, lozenges, aqueous or oily suspensions, dispersible powders or granules, emulsions, hard or soft capsules, or syrups or elixirs.

Tablets contain the nutritional supplement in admixture with non-toxic pharmaceutically acceptable excipients that are suitable for the manufacture of tablets. These excipients can be for example, inert diluents, such as calcium carbonate, sodium carbonate, sodium citrate, lactose, calcium phosphate, sodium phosphate, microcrystalline cellulose, corn starch, potato starch, and cellulose esters such as cellulose acetate, ethyl cellulose; granulating and disintegrating agents, for example, corn starch, or alginic acid, or complex silicates; binding agents, for example starch, polyvinylpyrrolidone, PEG-8000, gelatin or gum acacia, and lubricating agents, for example magnesium stearate, stearic acid, sodium lauryl sulfate, or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the nutritional supplement is mixed with an inert solid diluent, for example, calcium carbonate, calcium phosphate or kaolin, or as soft gelatin capsules wherein the nutritional supplement is mixed with water or an oil medium, for example peanut oil, liquid paraffin or olive oil.

Aqueous suspensions contain the nutritional supplement in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the nutritional supplement in a vegetable oil, for example arachis oil, olive oil, sesame oil or coconut oil, or in a mineral oil such as liquid paraffin. The oily suspensions can contain a thickening agent, for example beeswax, hard paraffin or acetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the nutritional supplement in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, preservative, flavoring, and coloring agents.

The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

Ingestion of the nutritional supplement of the present invention, or compositions comprising the nutritional supplement and one or more carriers and/or excipients has also been surprisingly found to be beneficial in the treatment of patients with kidney stones, gout and arthritis.

The effective amount of nutritional supplement that can be combined with the carrier and/or excipients to produce a single dosage form varies depending upon the host treated and the particular mode of administration. Dosage unit forms generally contain between from about 5 mg to about 5000 mg of the nutritional supplement. Doses of up to 1000-5000 mg are suitable for daily consumption. The term “effective amount” as used herein refers to an amount of the nutritional supplement sufficient to alleviate or reduce the symptoms of mineral deficiency, in particular calcium deficiency, or the symptoms of arthritis, kidney stones, and/or gout in the subject patient relative to the patients symptoms prior to administration of the nutritional supplement. It is understood that the effective amount for any particular patient or subject depends upon a variety of factors including the age, body weight, general health, sex, diet, time of administration, route of administration, and rate of excretion, and the severity of the particular condition undergoing treatment.

In the treatment of kidney stones the preferred dose is 230 mg of the nutritional supplement, to be taken daily over a three day period. For the treatment of other ailments a preferred dose is 800 mg taken 2-3 times per day.

Oral or parenteral administration of an effective amount of the nutritional supplement either alone or formulated as the compositions of the present invention relieves the symptoms of a person suffering from the abovementioned ailments. Typically, clinical patients suffering from kidney stones show a reduction of symptoms within 24 hours after oral or parenteral administration of the composition, with little or no adverse side effects. It is thought that the composition effects dissolution of the kidney stone which then passes through the patient in the conventional manner.

The following examples are provided to further illustrate several embodiments of the invention.

### Example 1

A bony deposit was removed from the skull of a cod fish, cleaned and ground to a fine whit powder. The powder was analysed and found to have a composition as set out in the following table.

| | ANALYSIS | | | | |
|---|---|---|---|---|---|
| | | | 3. | | Food Standards Code |
| | | 2. | Adult Dose 2 | | Maximum values for |
| | | Adult dose 1 | 400 mg (taken | | “Foods not containing |
| | 1. | 230 mg to be | three times | | a food not otherwise |
| | Results | taken daily | daily) | ×3 | standardised |
| Calcium, Ca mg/kg | 347,000 | 79.8 | 138.8 | 416 | — |
| Phosphorus, P ppm | 120 | 0.03 | 0.05 | 0.14 | — |
| Sodium, Na ppm | 1850 | 0.43 | 0.7 | 2.2 | — |
| Magnesium, Mg mg/kg | 14 | 0.0003 | 0.0006 | 0.02 | — |
| Cadmium, Cd ppm | <0.05 | Trace | Trace | Trace | 0.05 |
| Mercury, Hg ppb | <10 | <0.002 | <0.004 | <0.012 | 0.03 |
| Lead, pb ppm | | | | | |
| Zinc, Zn ppm | <1 | Trace | Trace | <0.001 | 1.5 |
| Chromium, Cr ppm | 3 | Trace | <0.001 | 0.004 | 150 |
| Copper, Cu ppm | <5 | <0.001 | <0.002 | <0.006 | N/A |
| | 1.5 | Trace | Trace | <0.002 | 10 |

As will be evident from the table, the first recommended dose of the nutritional supplement (see Column 2) is 230 milligrams taken daily over a three day period. The second recommended dose is the consumption of 400 mg taken three times per day (see Column 3). At these levels, beneficial amounts of calcium are ingested daily by the patient.

On the other hand the amounts of other components, in particular lead, cadmium, mercury, chromium, zinc and copper are ingested at well below the minimum amounts recommended in the Food Standards Code as shown by Column 4.

It is to be understood that, although prior art use and publications may be referred to herein, such reference does not constitute an admission that any of these form a part of the common general knowledge in the art, in Australia or any other country.

In the description of the invention, except where the context requires otherwise due to express language or necessary implication, the words “comprise” or variations such as “comprises” or “comprising” are used in an inclusive sense, i.e. to specify the presence of the stated features, but not to preclude the presence or addition of further features in various embodiments of the invention.

Numerous variations and modifications will suggest themselves to persons skilled in the relevant art, in addition to those already described, without departing from the basic inventive concepts. All such variations and modifications are to be considered within the scope of the present invention, the nature of which is to be determined from the foregoing description.

1. A nutritional supplement derived from skeletal tissue found in bony fish when used for the treatment of a patient with arthritis, kidney stones, and/or gout.

2. The nutritional supplement as defined in claim 1, wherein the skeletal tissue is found in a skull of the bony fish.

3. The nutritional supplement as defined in claim 2, wherein the skeletal tissue is configured as a pair of small deposits residing in the top of the skull.

4. The nutritional supplement as defined in claim 3, wherein the skeletal tissue is an otolith.

5. The nutritional supplement as defined in claim 1, wherein the skeletal tissue is found in the skull of bony fish from the Gadidae family.

6. The nutritional supplement as defined in claim 1, wherein the skeletal tissue is found in bony fish in a groups of families comprising: Elopidae, Albulidae, Chanidae, Meglopidae, Chirocentridae, clupeidae, Engraulidae, Muraenidae, Congridaw, Ophichthidae, Ariidae, Plotosidae, Synodotidae, Harpodontidae, Batrachoididae, Gobiescosidae, Antennariidae, Ophidiidae, Carapidae, Bythitidae, Exocoetidae, Hemirhamphidae, Belonidae, Atherinidae, Holocentridae, Monocentridae, Zeidae, Caproidae, Veliferidae, Fistularidae, Aulostomidae, Hoplichthidae, Dactylopteridae, Pegasidae, Centriscidae, Solenostomidae, Syngnathidae, Syngnathidae, Scorpaenidae, Aploactinidae, Triglidae, Platycephalidae, Serrandae, Grammistidae, Acanthoclinidae, Plesiopidae, Pseudochroraidae, Centropomidae, Ambassidae, Glaucosomidae, Teraponidae, Sillaginidae, Priacanthidae, Apogonidae, Carangidae, Coryphaenidae, Malacanthidae, Pomatomidae, Rachycentridae, Echeneidae, Menidae, Leiognathidae, Lobotidae, Lutjanidae, Haemulidae, Caesionidae, Banjosidae, Lethrinidae, Sparidae, Menipteridae, Gerreidae, Mullidae, Scianidae, Monodactylidae, Leptobramidae, Pempheridae, Kyphosidae, Scorpidadae, Toxotidae, Scatophagidae, Ephippididae, Rhinoprenidae, Chaetodontidae, Pomacanthidae, Pomacentridae, Pentacerotidae, Cirrhitidae, Creediidae, Cepolidae, Congrodadidae, Notograptidae, Mugilidae, Polyneraidae, Sphyraenidae, Labridae, Scaridae, Mugiloididae, Opistognathidae, Uranoscopidae, Blenniidae, Tripterygiidae, Callionumidae, Gobiidae, Eleotridae, Gobiodae, Zanclidae, Siganidae, Acanthuridae, Istiophoridae, Xiphiidae, Scombridae, Psetodidae, Bothidae, Pleuronectidae, Soleidae, Cynoglossidae, Percophididae, Centrolophidae, Nomeidae, Ariomraatidae, Triodontidae, Triacanthodidae, Triacanthidae, Balistidae, Monacanthidae, Ostraciidae, Tetraodontidae, Diodontidae.

7. The nutritional supplement according to claim 1, wherein the nutritional supplement is prepared by cleaning and grinding the skeletal tissue to a fine powder.

8. The nutritional supplement according to claim 1, wherein the nutritional supplement contains 300,000 to 400,000 mg/kg calcium, 100 to 150 mg/kg phosphorous, 10 to 20 mg/kg magnesium, and 1,500 to 2,000 mg/kg sodium.

9. A nutritional supplement composition comprising the nutritional supplement as defined in claim 1 a carrier and/or excipient.

10. A pharmaceutical composition comprising the nutritional supplement as defined in claim 1 and a pharmaceutically acceptable carrier and/or excipient.

11. A method of treating a patient with arthritis, kidney stones, and/or gout comprising administering an effective amount of the nutritional supplement as defined in claim 1 to a patient in need of such treatment.

12. The method of claim 11, wherein administering an effective amount of said nutritional supplement, said nutritional supplement composition, or said pharmaceutical composition comprises orally or parenterally administering an effective amount of the said nutritional supplement, said nutritional supplement composition, or said pharmaceutical composition to said patient.

13. The methods of claim 11, wherein the patient is administered a dosage in a range from about 5 mg to about 5000 mg of the nutritional supplement, nutritional supplement composition, or pharmaceutical composition, respectively on a daily basis.

14. The methods of claim 13, wherein the administered daily dose is up to 1000 mg-5000 mg.

15. A use of a nutritional supplement as defined in claim 1 in the manufacture of a medicament for the treatment of a patient with arthritis, kidney stones, and/or gout.
